(19) United States
(12) Patent Application Publication
(10) Pub. No.: US 2010/0331833 A1
(43) Pub. Date: Dec. 30, 2010

(54) IN-VITRO DEVICE MONITORING DURING MINIMALLY INVASIVE ABLATION THERAPY

(76) Inventors: Michael Maschke, Lonnerstadt (DE); Gudrun Zahlmann, Neumarkt (DE); Sebastian Schmidt, Weisendorf (DE); Karsten Hiltawsky, Schwerte (DE)

Correspondence Address: BRINKS HOFER GILSON & LIONE, P.O. BOX 10395, CHICAGO, IL, 60610, US

(21) Appl. No.: 12/492,315

(22) Filed: Jun. 26, 2009

(51) Int. Cl. A61B 18/02; A61B 18/14; A61N 7/00; C12Q 1/00; C40B 30/00

(52) U.S. Cl. 606/20; 606/42; 601/2; 435/4; 506/7

(57) ABSTRACT

A method and apparatus for monitoring and controlling interventional therapy is described, where the interventional therapy results in the destruction of tissue and the release of chemical markers. When interventional therapy results in destruction of a patient tissue, such as cancerous tissue, a variety of chemical markers may be released into the bloodstream. An in-vitro diagnostic (IVD) device may be used to measure the concentration of an analyte representing a marker and compare the measured concentration with a predetermined set point so as to monitor or control the procedure. The blood for the analysis may be obtained either inter-procedurally or intra-procedurally depending on the promptness of the effect being measured and the speed of measurement.

### TECHNICAL FIELD

The present application relates to a system and method of monitoring or controlling an interventional therapy. In particular the progress of the therapy is measured by an in-vitro diagnostic device.

### BACKGROUND

The use of ablation therapy for treating the occurrence of tumors is increasing. Ablation therapy is also useful in treating heart arrhythmias. The extent of tissue destruction and differentiation between tissue types is not well measured at present, leading to instances of excessive tissue destruction, including incidental destruction of healthy tissue, or insufficient tissue destruction, such that the treatment is not fully effective. Depending on the outcome of the ablation there will be a reduction of the tumor or the reduction of arrhythmias and an improved quality of life. However, what is unknown today during such procedures is the stage of cell death, and the extent thereof.

Ablation therapies apply a local physical “stress” to the tissue. Ablation therapies include laser therapy, where a laser is used to produce local heating, radio frequency ablation, electro-coagulation, high-intensity focused ultrasound (HIFU) electroporation (short high-voltage pulses), cryotherapy, and the like. The therapy is intended to damage the targeted tissue so that necrosis occurs or apoptosis is induced. A concern in performing ablation therapies or similar therapies is monitoring of the amount of ablation during the therapy, to provide the proper amount of cell death to specific tissue and not to damage the healthy surrounding tissue.

At present, control of the amount of ablation being performed is mostly based on trial and error, indirect measurements, or the experience of the physician. For an example, see US 2007/0066968, “Temperature Probe for Insertion into the Esophagus” which discloses a method to measure the temperature in the proximity of the atrium during an ablation procedure in the heart.

Imaging modalities such as X-ray (e.g. computed tomography (CT), magnetic resonance imaging (MRI), or ultrasound for ablation therapy monitoring has also been reported. CT and MRI are not practical for continuous monitoring or frequent monitoring due to the duration of the ablation procedure. When ultrasound is used for monitoring an ablation process, the process itself is monitored, mainly due to the resulting air bubbles. But, the differentiation between healthy tissue and cancerous tissue during the ablation therapy is difficult.

Using MRI, methods are known to monitor the temperature of the tissue during ablation, which gives a rough outline of the zone that reaches the critical temperature. However, the method is complex and error-prone. Other methods of control use numerical simulations to simulate the heat distribution within the tissue and to calculate the amount of energy that is required to achieve the purpose of the therapy. This is may be done based on previously acquired images of the patient. However, these methods often do not provide sufficiently accurate or consistent results, because of mismatches between the model and the physiological situation.

In-vitro diagnostic (IVD) products are those reagents, instruments, and systems intended for use in diagnosis of disease or other conditions, including a determination of the state of health, in order to cure, mitigate, treat, or prevent disease or its sequelae. Such IVD products are intended for use in the collection, preparation, and examination of specimens taken from the human body. (21 CFR 809.3)

Bio-markers and other in-vitro diagnostic tests are becoming available to assist in the diagnosis. A panel of markers may be needed in order to achieve clinically acceptable sensitivity and specificity for a particular purpose device.

A blood analysis device such as “Lab on a Chip”, which is being developed by Siemens AG, may be used for determining blood values or certain genetic or molecular markers (see, for example, WO 00/56922, “Genetic Polymorphism and Polymorphic Pattern for Assessing Disease Status, and Compositions for Use Thereof”, and WO 00/23802, “Method for_Measuring Cellular Adhesion” for gene tests and tests with molecular markers for stroke). See also, WO 2005/106024, entitled “method and Assembly for DNA Isolation with Dry Reagents” and WO 2005/106023, entitled “PCR Process and Arrangement for DNA Amplification using Dry Reagents.” All of the above references are incorporated herein by reference as examples of devices and methods which may be used in IVD.

### SUMMARY

A method of monitoring or controlling an interventional procedure is described, including establishing a procedure-specific analyte and a set-point value thereof and obtaining a sample of a bodily fluid. The interventional procedure is performed and the bodily fluid is obtained either intra-procedurally or inter-procedurally. The value of the analyte in the bodily fluid is determined and compared with the set-point value.

In an aspect a system for interventional treatment includes an interventional apparatus capable of delivering or removing localized energy to a location in a patient body; and an in-vitro diagnostic (IVD) device. A bodily fluid is analyzed by the IVD device to determine a value of an analyte, and the value of the analyte is compared with a pre-determined value to control a value of the localized energy.

In another aspect, a computer program product, stored on a machine readable medium, includes instructions for configuring a computer to: accept a measured value of an analyte from an in-vitro diagnostic (IVD) device; compare the measured value of the analyte with a stored value, and control the energy delivered to, or removed from, a patient by an interventional device, based on a relation between the measured value and the stored value.

### BRIEF DESCRIPTION OF THE DRAWINGS

FIG. 1 is a block diagram of a system for delivering interventional therapy to a patient while monitoring or controlling the therapy using an in-vitro diagnostic (IVD) device;

FIG. 2 shows steps in a first example of a method;

FIG. 3 shows steps in a second example of a method;

FIG. 4 shows steps in a third example of a method;

FIG. 5 shows steps in a fourth example of a method; and

FIG. 6 shows steps in a fifth example of a method.

### DETAILED DESCRIPTION

Exemplary embodiments may be better understood with reference to the drawings. In the interest of clarity, not all the routine features of the implementations described herein are described. It will of course be appreciated that in the development of any such actual implementation, numerous implementation-specific decisions must be made to achieve a developers' specific goals, such as compliance with system and business related constraints and regulatory requirements, and that these goals and constraints will vary from one implementation to another.

An IVD test for apoptosis or necrosis may be employed during an ablation therapy session, or between therapy sessions, so as to measure the progress and effectively of an ablation therapy procedure. For each therapy procedure, a level of specific panel of IVD markers is selected so that the test result may be used to control the therapy procedure or session so that the desired extent of cell death through necrosis or apoptosis indicates a specific level or threshold of cell death is achieved. The specific panel of markers and the selected concentration levels would be selected, for example, based on the type of cells to be killed, and the mass of cells. A specific tissue type to be protected may also be identified.

Accordingly, the specific panel of markers would be developed by clinical or experimental studies so as to be most appropriate for the specific syndrome to be treated.

During necrosis or apoptosis cells release markers that, in a healthy cell are normally located intracellular. The presence and concentration of specific markers may be indicative of the amount and type of cells that are being damaged or killed.

US 2002/0177120, “Assays for Apotosis Modulations” discloses a method for screening assays for identifying and selecting candidate compounds modulating apoptosis. U.S. Pat. No. 6,964,854, “Compositions and Methods Useful for the Diagnosis and Treatment of Heparin induced Thrombocytopenia/Thrombosis” and US 2008/0050832, “Methods and Composition for Diagnosis and/or Prognosis in Systemic Inflammatory Response Syndromes” disclose methods to indicate necrotic processes in cells. The above patent documents are included into this invention disclosure by reference.

Possible markers for the purpose of monitoring the ablation therapy include: fragmented free nucleic acids (DNA, RNA), which can be measured by immunoassays; intracellular enzymes such as creatine kinase, which can be measured by immunoassays or clinical chemistry assays; intracellular other proteins such as troponin, which can be measured with immunoassays; intracellular electrolytes such as potassium, which can be measured with specific electrodes; and, pH changes, which can be measured with specific electrodes. Another marker may include proteins denatured by heat, which form high-molecular weight complexes with reduced solubility, and which can be detected by gel electrophoresis or immunoassays. Monoclonal antibodies (MAbs) to specific tumor surface sugars can also be used as markers.

In a first example, a selected therapy technique is applied in several sessions (fractions) and blood is drawn from the patient at a fixed time after each therapy session. The measured level of the appropriate IVD marker is then used to estimate amount of cell death that has recently occurred and plan the next session(s). This may termed “inter-fractional monitoring”.

Herein, except where specifically indicated, the use of the term “cell death” is intended to encompass any cause of the destruction of a specific cell type by the therapy, whether such cell death is immediate or to be expected as a delayed result of the therapy. That is, both necrosis and induced apoptosis, either separately or in combination, may be measured. Moreover, individual cell type may also be identified, e.g., cancerous or non cancerous, by one or more of the markers used in the IVD test panel selected.

In another example the amount of the analyte is monitored during the session: for example, by taking several samples of blood sequentially and analyzing them quickly, such as with an electrolyte-selective electrode. The measured amount of the analyte is then used as a feed-back parameter to control the therapy during the session. An example is a laser therapy such as laser induced thermotherapy (LITT), where the intensity of the beam may be adjusted to keep the analyte concentration within a target range.

Herein, the term “analyte” means a substance, chemical constituent, or component of a sample from a patient that is identified or quantified in an analytical procedure. For instance, in an immunoassay, the analyte may be a ligand or binder, while in blood glucose testing, the analyte is glucose.

An analyte itself may not be measured, but a measurable property of the analyte can be measured. For example, glucose may not be measured, but the concentration of glucose can be measured. In this example “glucose” would be the component of the sample and concentration is a property of the glucose component. Commonly, the term “property” is omitted, provided the omission does not lead to an ambiguity as to what property of the sample is being measured.

In another example, the analyte may be measured by the IVD device as a safety measure. Once the analyte concentration for one or more specific components reaches a certain threshold, the therapy is stopped or the applied energy in an ablation procedure is reduced, so as to avoid collateral damage.

In yet another example, blood samples for the IVD analyte may be collected using a catheter that is placed in a vein coming from the region being treated. Alternatively, the IVD measurement device may be completely or partially placed within the body of the patient.

In still another example, the expected amount of analyte is first estimated in a simulation model. Where the therapy is simulated by calculation of the tissue volume that intended be destroyed, and the resultant concentration of, or the cumulative amount of, analyte that is expected to result is determined. The analyte panel may, as in other examples, include markers for the specific type of tissue to be destroyed, and for monitoring of markers for tissue that is desired to be preserved. The analyte levels are compared to the expected levels for each particular therapy plan, and the therapy may be adjusted or terminated accordingly.

Depending on the specific IVD device, the measured analyte properties may be used for real-time, that is, intra-procedural, feedback as to the progress of the procedure. For example, if a specific tumor cell marker is being monitored, where the tumor cell marker is released during the therapy due to cell death of tumor cells, this information can be used to continue the therapy. If such a specific tumor cell marker concentration falls below a selected level, the procedure would be stopped, as healthy cells may be damaged, as an insufficient mass of tumor cells may remain in the treatment volume.

Further, specific markers can be used, in order to detect injury to major vessels, and excess heat which may cause “cooking” of blood instead of cellular tissue. Blood parameters, which are known to change in case of a haemolysis, e.g., haptoglobin or pH, may be used so as to monitor the therapy, and the therapy may be discontinued when inappropriate markers of concentrations of markers are found.

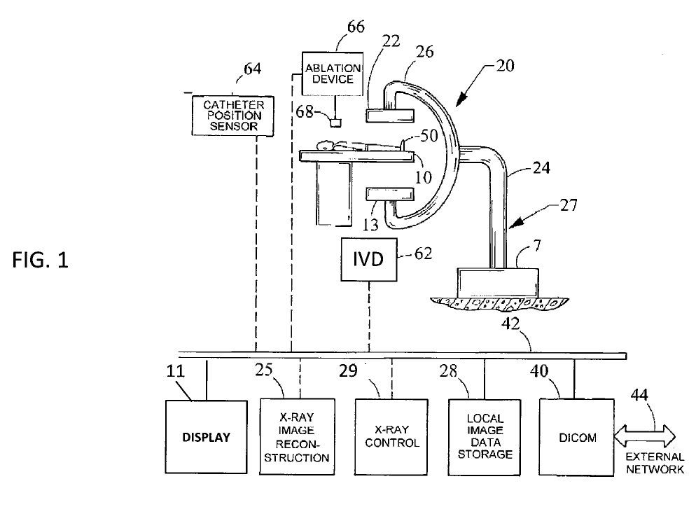

FIG. 1 shows a block diagram of an example of a system for the diagnosis and treatment of an illness by a use of a catheter to perform ablation therapy. Other embodiments of the system may include fewer than all of the devices, or functions, shown in FIG. 1. It will be understood by persons of skill in the art that the signal and data processing and system control is shown in an example, and that many other physical and logical arrangements of components such as computers, signal processors, memories, displays and user interfaces are equally possible to perform the same or similar functions. The particular arrangement shown is convenient for explaining the relationship of the elements and the functionality of the system.

A C-arm X-ray device 20 is representative of an imaging modality which may be used and comprises a C-arm support 26 to which an X-ray source 22, which may include a diaphragm to limit the field of view, and an X-ray detector 13 may be mounted so as to face each other along a central axis of radiation. The C-arm 26 is mounted to a robotic device 27 comprising a mounting device 7, and one or more arms 24 which are articulated so as to be capable of positioning the C-arm X-ray device with respect to a patient support apparatus 10.

The C-arm X-ray device 20 is rotatable such that a sequence of projection X-ray images of a patient 50 is obtained, and the images are reconstructed by any technique of processing for realizing computed tomographic (CT-like) images. Alternatively, the C-arm X-ray device may be used to obtain real-time (fluoroscopic) images for guiding an interventional device, such as a catheter 68, which may be an ablation catheter.

The IVD device 62 may be mobile or fixedly installed. In an example, a “point-of-care” device positioned in proximity to the patient, permits a patient bodily fluid to be drawn and placed in the IVD device. US 2008/0312519 describes a mobile examination unit having an integrated set of sensors based on a “Lab-on-a-chip” such as described in DE 10 2004 021 780 and DE 10 2004 822 A mobile analyzer can serve several treatment rooms and may be moved to the one where a treatment procedure is currently being performed. Another kind of point of care device, which could be installed in each treatment room, as disclosed in US 2005/0123444, is desktop analyzer. Another point-of-care IVD is disclosed in U.S. Pat. No. 6,845,327 which may have analytic sensors in each treatment room to obtain the body fluid sample and perform a pre-analysis, where a final analysis performed on a central computer accessed over a communications network, which may be a local area network (LAN).

The most effective analyzer, from a physician's viewpoint, may be an IVD can be positioned close to the patient and give an immediate result in the room.

Alternatively, depending on the equipment available, is also possible to use the more centralized kind of body-fluid analyzers where, for example, a blood sample is drawn in a small container, which is sent down to a central lab and the results are send back by FAX or other means. Although the specific tests that would be performed by such an analyzer may differ from therapy type to therapy type, a person of skill in the art would be capable of selecting an appropriate analyzer or request the development of an analyzer suitable for obtaining the required data.

The devices and functions shown in FIG. 1 are representative, but not inclusive. Other imaging modalities such as MRI, CT, US and the like, either individually or in combination. The individual units, devices, or functions may communicate with each other over cables or in a wireless manner, and the use of dashed lines of different types for some of the data and control connections in is intended to suggest that alternative means of connectivity may be used.

The C-arm X-ray radiographic device 20 and the associated image processing 25 may produce angiographic or soft tissue computed tomographic images comparable to, for example, CT equipment, while permitting more convenient access to the patient 50 for ancillary equipment such as the catheter 68, and treatment procedures. A separate processor 25 may be provided for this purpose, or the function may be combined with other processing functions.

Images reconstructed from the X-ray data may be stored in a non-volatile (persistent) storage device 28 for further use. The X-ray device 20 and the image processing attendant thereto may be controlled by a separate controller 29 or the function may be consolidated with the user interface and display 11.

The treatment device may be an ablation tool 66 having a catheter 68 which is introduced into the body of the patient 50 and guided to the treatment site by images obtained by the C-arm X-ray, or other sensor, such as a catheter position sensor 64. The catheter position sensor may use other than photon radiation, and electromagnetic, magnetic and acoustical position sensors are known.

The various devices may communicate with a DICOM (Digital Communication in Medicine) system 40 and with external devices over a network interface 44.

Some or all of the signal and data processing and data display may also be located in the treatment room; however, equipment and functionality not directly related to the sensing or manipulating of the patient or the interventional device, may be remotely located. Such remote location may be facilitated by high speed data communications on local area networks, wide area networks, and the Internet. The signals representing the data and images may be transmitted by modulation of representations of the data on electromagnetic signals such as light waves, radio waves, or signals propagating on wired connections.

The system sensors, such as the IVD device 62 and the X-ray device 20 may thus be located remotely from the specialists making the diagnosis and for determining or administering the appropriate course of treatment. Of course, the specialists may be present with the patient at times as well.

A catheter locating system (see, for example, U.S. Pat. No. 5,042,486, “Catheter Locatable with Non-Ionizing Field and Method for Locating Same”) for the ablation catheter can be integrated into the system. The catheter 68 may be provided with position sensors, such as electromagnetic sensors or ultrasound-based sensors. Thus the tip of the ablation catheter, in particular, can be detected without emitting continuous X-rays and the motion thereof can be followed and displayed with respect to a previously obtained image.

In another alternative, an Acunav catheter (ultrasound catheter) can be used with 3D ultrasound images in real time for guiding the ablation catheter. (see, for example, U.S. Pat. No. 6,923,768, “Method and Apparatus for Acquiring and Displaying a Medical Instrument Introduced into a Cavity Organ of a Patient to be Examined or Treated”).

A treatment suite having at least a treatment device and an IVD device configured to perform analyte measurements appropriate for the treatment to be performed may be used so to perform one or more of the methods described herein.

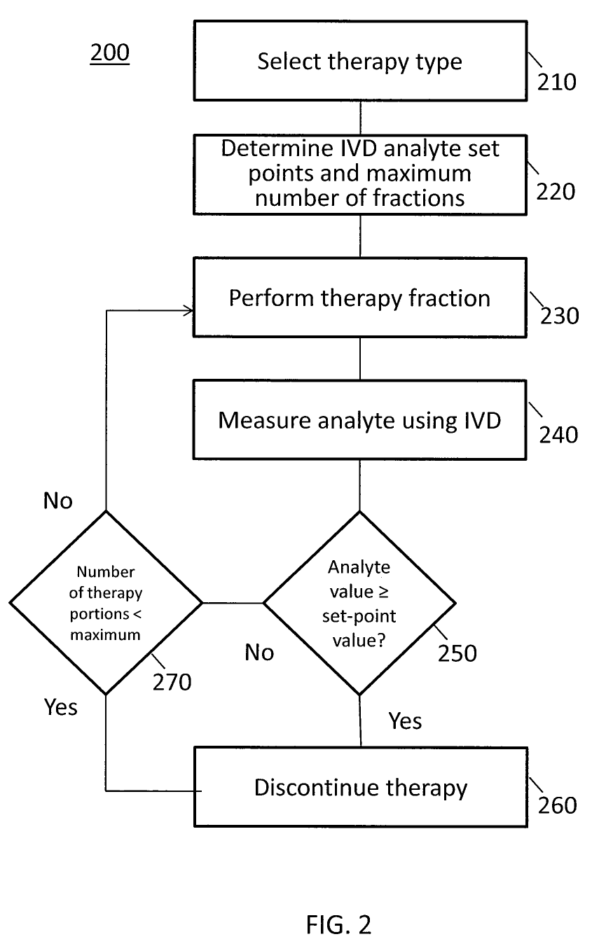

In a first example 200 of a method of monitoring and controlling a interventional therapy, shown in FIG. 2, a specific therapy is chosen (step 210) to treat a patient syndrome. Each individual patient will have had a diagnosis of a condition that may be treated interventional, such as by ablation therapy. Depending on the syndrome to be treated, patient specific data such as, size of the tumor, or location of an area of the heart responsible for arrhythmia, the therapy, which may be ablation therapy, is intended to destroy tissue, either directly or during a time after application of the energy of the therapy, the tissue that is the target of the intervention. For each type of syndrome and interventional procedure there may exist analytes which are a measure of the amount of tissue being destroyed, either by necrosis occurs or induced apoptosis. A particular panel of analytes, appropriate to the tissue being destroyed, and the therapy type is selected (step 220) including absolute values of the analytes indicating sufficient tissue destruction. Relative values of a plurality of analyte may also be used.

While the description of the methods herein uses a catheter and ablation as the method of treatment, other methods may be used where the necrosis or induced apoptosis is caused by the injection of a substance, the use of targeted radiation or focused ultrasound.

Once the appropriate IVD analyte panel is selected, the patient may be prepared for the therapy in accordance with local hospital procedures, which may vary to some extent due to regional or national regulations or practice. In an aspect, the treatment may be performed in increments or sessions, which may be called “fractions” (step 230). That is, only a portion of the tissue may be affected by the energy delivered during a fraction of the therapy. A sample of the bodily fluid, such as blood, may be collected or accessed so that the IVD panel of analytes may be quantified (step 240). This may be accomplished by drawing blood manually or automatically, and analysis in the therapy room or by a central laboratory, depending on the specific IVD and analyte panel in use. It follows therefore, that this method may be performed while the patient remains in a treatment room, if the IVD analysis is rapid, or in multiple individual sessions if the analysis takes some time. This time interval may be due to either the time to perform the IVD, or the time that may need to elapse before the effect of the therapy is apparent in an analyte.

The analyte value is compared with the pre-established set point value for the specific patient (step 250) and either the treatment is continued if the set point value is not reached, and the number of fractions is less than the total number of planned fractions (step 270), or the treatment is terminated if the analyte exceeds the set point value (step 260).

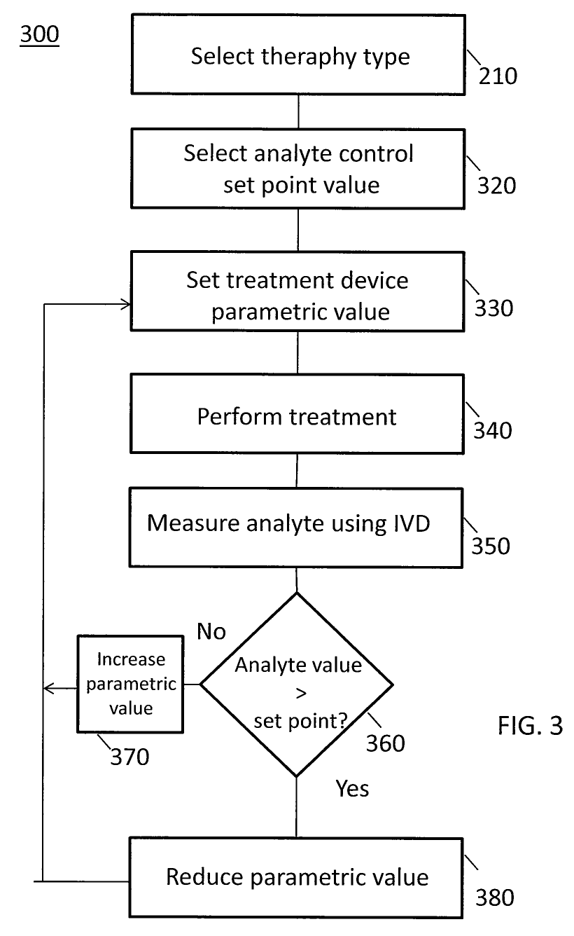

In a second method 300, shown in FIG. 3, an intensity of the treatment is controlled based on IVD measurements of one or more analytes. The first step (step 210) is shown as being the same as in the first method 200, in the sense that, for each treatment, a IVD analyte panel is chosen, and the values of analyte used as set point values is selected based on the objective of the treatment. Step 210 therefore is intended to be generic; however, the result of performing the step is that the specific IVD analyte panel and set points are treatment and patient specific.

A IVD set point value is selected that may be indicative of a particular rate of cellular destruction (step 320). The treatment is initiated at an energy level that is known to be safe for the patient (step 330). A maximum safe value for the laser intensity, radio frequency heating energy, sound intensity, or the like, is established. This value is not to be exceeded during the treatment. In an aspect, the total energy administered to the patient allowable during the treatment may also be established, so that a combination of the intensity and the time of application is used as an upper limit on energy dose. These limits are intended to be safety limits so that the patient is not exposed to either an intensity or total energy that would cause unintended harm.

The laser, or other device is set to an initial intensity (step 330) which may initially be a pre-determined fraction of the maximum intensity, so as to commence the treatment (step 340). In this example, the analyte may be measured (step 350) during the course of the treatment step 330 so as to monitor the effectivity of the initial intensity of treatment. The measured analyte is compared with the predetermined set point value so as to determine if the current rate of tissue destruction is greater than or less than the pre-planned value (step 360). Generally, the first measured values will be less than the pre-planned value as the treatment may begin at an intensity which is low compared with the capability of the laser or other treatment device. When the measured analyte is less than the pre-planned value, (step 370), the intensity value is increased (step 330) and the treatment continued.

As there may be a hysteresis effect, where the measured analyte value is representative of the effect of the treatment at an earlier time, the change in intensity value may be moderated so that, for example, the change in intensity is a fixed percentage of the difference between the maximum intended intensity and the current intensity. Alternatively the change in intensity may be proportional to the ration of the measured analyte value to the pre-planned value.

The maximum planned value for the analyte may take the hysteresis effect into account so that the maximum rate of intended tissue destruction is not exceeded. Alternatively, providing that the increments of intensity are chosen to avoid overheating, the analyte value may exceed that pre-planned value (step 380), and the intensity value may be decreased (step 380) and the process continued. The criteria for terminating the treatment may be that the total energy delivered to the patient has reached the pre-planned maximum value. Alternatively, a variety of the techniques described with respect to the other methods may be used. For example, the total analyte concentration, an analyte value indicating damage to healthy tissue, or an analyte value indicating that the pre-planned intensity is not producing a sufficient rate of destruction may be used individually or in combination so as to terminate the therapy session.

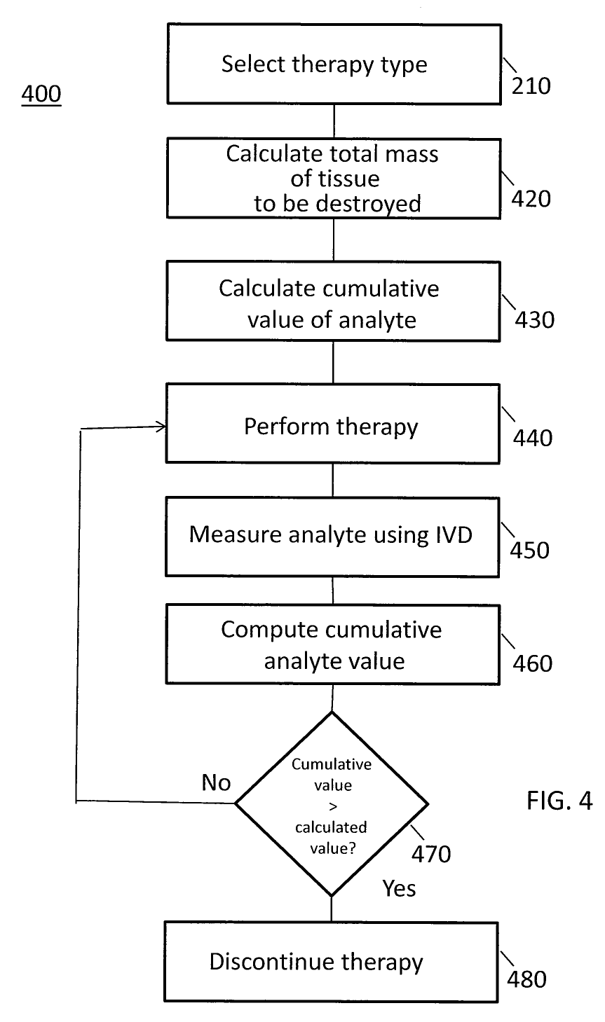

In a third example of a method 400, shown in FIG. 4 of using IVD for monitoring or controlling an interventional therapy, an IVD analyte panel is chosen, and the values of analyte used as set point values is selected based on the objective of the treatment (step 210). Here, the IVD is used to monitor the total amount of tissue of the specified type that is intended to be destroyed by the treatment, so that the treatment can be ended before additional healthy tissue is destroyed. The preparations for the treatment and general details of the treatment would be understood to be comparable to that previously described. For this and subsequent method descriptions, the description focuses on the additional steps that may be performed. However, the additional steps or description provided should not be interpreted as suggesting that the steps or description cannot or should not be combined with the steps of another method described herein.

In a treatment where a specific amount of identified tissue type is intended to be destroyed, the total amount of tissue to be destroyed (step 420) may be related to a total amount of a specific analyte, which is known to be a proxy for the target tissue. More than one analyte may be used as such a measure, and a ration of two or more analytes may also be used. Set point values corresponding the cumulative amount of an analyte may be computed, so as to establish a measurable proxy for the tissue to be destroyed (step 430). Although not shown, additional computations may be made regarding measures of the rate of tissue destruction, and such measures may be used as a further monitoring aspect of the method. For example if the rate of tissue destruction per unit of applied energy falls below a preset limit, or the rate of destruction of healthy tissue exceeds a preset limit, the therapy may be discontinued.

The performance of the treatment (step 440) is commenced. The selected analyte(s) are measured either intra-procedurally, or between treatment fractions, depending on the speed of the IVD analysis, and the time needed for the selected analyte markers to be representative of the desired treatment effect (step 450). The cumulative amount of analyte is computed (step 460) and compared with the pre-set value (step 470). If the cumulative analyte value is less than the pre-set value, then the process returns to step 440 and the treatment continues. Near the end of the treatment process, where the cumulative value of analyte becomes almost equal to the pre-set value, the intensity of the treatment may be reduced, or the duration of the application may be reduced, so as to minimize the possibility of over-treating the patient. When the cumulative value of the analyte has reached the pre-set value, the treatment is discontinued (step 480).

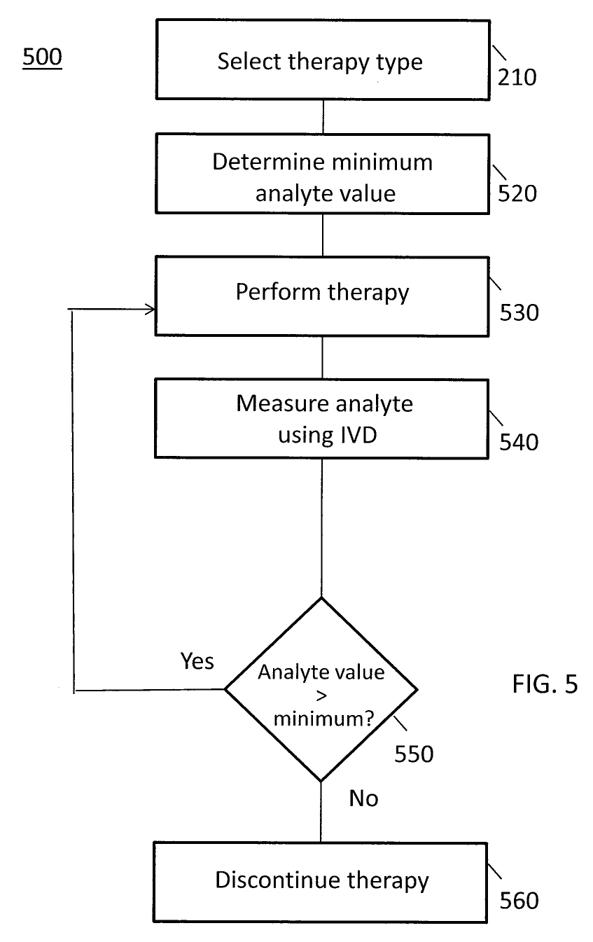

In fourth example of a method 500 of monitoring or controlling a treatment, shown in FIG. 5, the generic step of selecting the therapy type (step 210) is performed. A minimum analyte value for a marker of tissue destruction is selected (step 520). This value may be expressed as equivalent a rate of tissue destruction per unit of energy delivered. That is, the target tissue to be destroyed is expected to be destroyed at a rate that is proportional to the energy applied. If the rate of destruction begins to fall below this expected value, the measurement may be indicative that the target tissue has been destroyed, and that healthy tissue may now begin to be destroyed.

The treatment is performed (step 530) and the analyte value is measured (step 540). The time history of the analyte during the procedure may be reviewed to determine if the analyte value is relatively steady at a value that is indicative of the rate of planned tissue destruction (step 550). If this criteria is met, then the treatment is continued (step 530), or if the criteria is not met, the treatment is discontinued (step 560). While the steps have been described herein as being sequential, it should be understood that treatment, measuring and controlling steps may be performed contemporaneously or simultaneously.

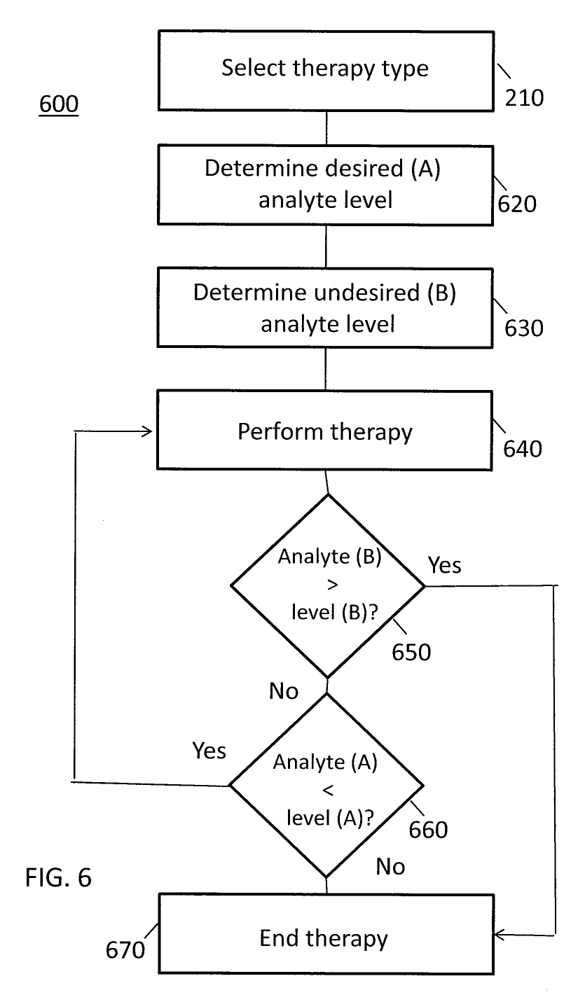

In fifth example of a method 600 of monitoring or controlling a treatment, shown in FIG. 6, the generic step of selecting the therapy type (step 210) is performed. This method may be considered to be a variation on the fourth method, where the rate of tissue destruction is used to determine the end point of the therapy. Here, an analyte (A) is selected, as before (step 620) so as to represent the tissue to be destroyed, and an analyte (B) is selected to represent tissue that is not to be destroyed (step 630). Alternatively, a ratio of analyte (A) to analyte (B) may be used.

The procedure is performed (step 630) and each of the analytes is successively evaluated. In step 650, the value of analyte (B) is compared against a predetermined set point so as to avoid excessive destruction of healthy tissue. If this set point is exceeded, the treatment is discontinued (step 670). If the value of analyte (B) is less that the set-point, then the value of analyte (A) is compared with the set point. If the value of analyte (A) is less than the set point, the procedure is continued (step 640), else the procedure is discontinued (step 670).

While the methods disclosed herein have been described and shown with reference to particular steps performed in a particular order, it will be understood that these steps may be combined, sub-divided, or reordered to from an equivalent method without departing from the teachings of the present invention. Accordingly, unless specifically indicated herein, the order and grouping of steps is not a limitation of the present invention.

It should be appreciated that as the use of IVD panels becomes more widespread, other treatments and other syndromes may be treated using the methods described herein.

The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

In an embodiment, the instructions may be stored on a removable media device for reading by local or remote systems. In other embodiments, the instructions may be stored in a remote location for transfer through a computer network, a local or wide area network, by wireless techniques, or over telephone lines. In yet other embodiments, the instructions are stored within a given computer, system, or device.

Where the term “data network”, “web” or “Internet” is used, the intent is to describe an internetworking environment, including both local and wide area networks, where defined transmission protocols are used to facilitate communications between diverse, possibly geographically dispersed, entities. An example of such an environment is the world-wide-web (WWW) and the use of the TCP/IP data packet protocol, and the use of Ethernet or other known or later developed hardware and software protocols for some of the data paths.

Communications between the devices, systems and applications may be by the use of either wired or wireless connections. Wireless communication may include, audio, radio, lightwave or other technique not requiring a physical connection between a transmitting device and a corresponding receiving device. While the communication is described as being from a transmitter to a receiver, this does not exclude the reverse path, and a wireless communications device may include both transmitting and receiving functions. There term “wireless communication” is understood to comprise the transmitting and receiving apparatus, including any antennas, and any modem used to encode or decode the data, speech, or the like, for transmission using electromagnetic waves.

Although only a few exemplary embodiments of this invention have been described in detail above, those skilled in the art will readily appreciate that many modifications are possible in the exemplary embodiments without materially departing from the novel teachings and advantages of the invention. Accordingly, all such modifications are intended to be included within the scope of this invention as defined in the following claims.

What is claimed is:

1. A method of monitoring or controlling an interventional procedure, comprising the acts of:
establishing a procedure-specific analyte and a set-point value thereof;
obtaining a sample of a bodily fluid;
performing the interventional procedure
determining a value of the analyte in the bodily fluid; and
comparing the analyte value with the set-point value.

2. The method of claim 1, wherein the interventional procedure is an ablation procedure.

3. The method of claim 1, wherein the procedure-specific analyte is a biomarker.

4. The method of claim 1, wherein the value of the analyte is determined by an in-vitro diagnostic device (IVD).

5. The method of claim 4, wherein the IVD uses a microfluidic device.

6. The method of claim 4, wherein the IVD uses a lab-on-a-chip.

7. The method of claim 4, wherein the IVD uses a DNA micro-array.

8. The method of claim 1, wherein the step of comparing the analyte value results in determining that the analyte value is less than the set-point value, the step of performing the interventional procedure is continued.

9. The method of claim 8, further comprising:
performing the interventional procedure in fractions, and the act of obtaining a sample of bodily fluid is performed after the act of performing each fraction.

10. The method of claim 8 wherein act of performing obtaining the sample of bodily fluid is performed at intervals during the interventional procedure.

11. The method of claim 1, further comprising:
performing the interventional procedure using a device having a controllable intensity.

12. The method of claim 11, further comprising controlling an initial intensity of the device to be below a predetermined safety limit.

13. The method of claim 12, wherein when the measured analyte is less than the set-point value, the intensity is increased, while controlling the intensity to remain lower than the safety limit.

14. The method of claim 12, wherein when the measured analyte is greater than the set point value, controlling the intensity to be decreased.

15. The method of claim 1, wherein the set-point value is proportional to a mass of tissue to be destroyed by the procedure, the method further comprising:
comparing the set point value with the cumulative value of the analyte from a start of the interventional procedure, and discontinuing the interventional procedure when the cumulative value is equal to the set point.

16. The method of claim 1, further comprising determining a minimum analyte value as a set point, the set point representing a minimum rate of tissue destruction, and discontinuing the treatment when the analyte value falls below the set point for a specific intensity.

17. The method of claim 1, wherein the set point comprises a first set point and a second set point, the first set point representing a value of a first analyte for which a criteria of destroying selected tissue is met, and a second set point representing a value of a second analyte for which a criteria of not destroying healthy tissue is met, and continuing the procedure when both the condition of destroying selected tissue and not destroying healthy tissue is met.

18. The method of claim 1, wherein the bodily fluid is blood.

19. The method of claim 18, wherein the blood is drawn from a vein proximal to the tissue being treated.

20. A system for interventional treatment comprising:
an interventional apparatus capable of delivering or removing localized energy to a location in a patient body;
an in-vitro diagnostic (IVD) device;
wherein a bodily fluid is analyzed by the IVD device to determine a value of an analyte, and the value of the analyte is compared with a pre-determined value to control a value of the localized energy.

21. The system of claim 20, wherein the interventional apparatus is an ablation tool.

22. The system of claim 21, where the ablation tool is one of a laser, a radio frequency heater, a high-intensity ultrasound device, or a cryogenic cooler.

23. The system of claim 20, wherein the analyte is a biomarker.

24. The system of claim 20, where the bodily fluid is blood.

25. A computer program product, stored on a machine readable medium, comprising:
instructions for configuring a computer to:
accept a measured value of an analyte from an in-vitro diagnostic (IVD) device;
compare the measured value of the analyte with a stored value and control the energy delivered or removed from a patient by an interventional device based on a relation between the measured value and the stored value.

26. The computer program product of claim 25, wherein the stored value represents pre-computed value determined as a set-point

27. The computer program product of claim 26, wherein the set-point is one of: a rate of destruction of targeted tissue, a total amount of targeted tissue destroyed, a minimum rate of destruction of targeted tissue per unit of energy, or a maximum rate of destruction of non-targeted tissue.
